(19) United States
(12) Patent Application Publication
(10) Pub. No.: US 2007/0083162 A1
(43) Pub. Date: Apr. 12, 2007

(54) Valve for intravenous catheter

(75) Inventors: James O'Reagan, Greer, SC (US); Theodore J. Mosler, Raleigh, NC (US)

Correspondence Address: DRINKER BIDDLE & REATH LLP;ATTN: PATENT DOCKET DEPT., 191 N. WACKER DRIVE, SUITE 3700, CHICAGO, IL, 60606, US

(73) Assignee: Span-America Medical Systems, Inc., Greenville, SC (US)

(21) Appl. No.: 11/247,070

(22) Filed: Oct. 11, 2005

(51) Int. Cl. A61M 5/178

(52) U.S. Cl. 604/167.03; 600/573

(57) ABSTRACT

The present invention is directed to a valve assembly in an intravenous catheter that facilitates the administration of fluid to a patient through the intravenous catheter by a needleless device. The valve assembly of the present invention contains means for providing a positive displacement of fluid from the catheter at a time when a needleless device is removed from the valve assembly following its connection to the valve assembly.

### FIELD OF THE INVENTION

The invention is directed to valves, and more specifically, to valves in medical devices that control fluid flow.

### BACKGROUND OF THE INVENTION

Intravenous catheters are medical devices for administering intravenous fluids, medications, and blood products. Intravenous catheters may also be used for aspirating blood for testing or donation. An intravenous catheter generally consists of a hollow-bore needle and a close-fitting, over-the-needle plastic catheter tubing used to access the lumen of a blood vessel in a patient. After the needle and catheter are inserted into the blood vessel, the needle is retracted from the patient and discarded, leaving only the catheter in the blood vessel. The catheter contains a catheter hub through which fluids, medications, and blood may be injected or through which blood samples may be taken from the patient. Needles were originally employed for accessing the catheter hub, but now needle-free injection sites or valves have been developed to eliminate the problems associated with the use of needles in medical procedures. Conventional catheter hubs now contain a valve wherein the outlet side of the valve is connected to the catheter.

Conventional valves contain a standard male-to-female medical luer-friction connection between the outlet side of a syringe or other device and the inlet side of the needle-free valve. When this connection is made, a piston in the valve is displaced from a closed position to an open position which allows fluid to flow through the valve to the output side of the valve. Once the fluid has been administered to the patient or the blood sample taken, the syringe or device can be disconnected from the valve and the piston returns to its closed position to seal the injection valve.

Conventional valves contain a space within which fluid flows from the syringe or other device to the catheter line on which the valve is mounted. When the syringe or other device is connected to the valve, it typically occupies a portion of, or changes the volume within the internal valve space, displacing the fluid (whether it be a liquid or air) within the valve. With many conventional valves, a problem arises when the syringe or device is disconnected from the valve. When the syringe or device is disconnected, the volume within the valve space increases. The increase in space within the valve results in fluid in the valve and catheter line moving to fill the space. In effect, the removal of the syringe or device creates a differential pressure in the flow path which in turn creates a suction force which draws fluid into the catheter. In the medical setting, this movement of fluid is very undesirable. When the valve is connected to a fluid line leading to a patient, the movement of fluid through the line towards the space in the valve has the effect of drawing blood from the patient in the direction of the valve. A serious problem may result in that this blood may clot and clog the catheter near its tip, rendering it inoperable, and may even result in a clot of blood being injected into the patient.

The risk of blood clogging the catheter is significantly increased in catheters having a small diameter (e.g., 24 gauge). Small catheters, however, reduce the trauma and discomfort caused by insertion of a catheter into a patient. Because these catheters have a very small internal passage, even a small suction force may draw a significant amount of fluid back through a catheter toward the valve, introducing blood into the catheter tip.

Fluids such as saline or heparin can be used to flush the flow path of the catheter tubing to prevent fluids and blood from being drawn back through the catheter tubing toward the valve. These fluids also serve to dilute any body fluids that would be drawn toward the valve. Saline and heparin, however, are not always available to flush the flow path when removing the syringe or device. Heparin is also often contraindicated for patient treatment. Finally, the use of saline or heparin does not provide a consistent solution to the problem because the user cannot be sure that the bodily fluids that were drawn toward the valve did not block the flow path, rendering the catheter unusable.

Other considerations effecting the design and operation of valves for intravenous catheters include maintaining sterility of the fluid and providing a smooth passage for the flow of fluids. Accordingly, a need exists for a needleless intravenous valve that does not cause blood from the patient to enter the catheter when a needleless injection device is removed from the valve; does not cause fluid to stagnate in the valve to compromise the sterility of the system; and does not damage blood products by having internally restrictive passageways.

### SUMMARY OF THE INVENTION

The invention is directed to a valve assembly in an intravenous catheter that facilitates the administration of fluid to a patient through the intravenous catheter by a needleless device. The valve assembly of the present invention contains means for creating a positive displacement of fluid from the intravenous catheter at a time when a needleless device is removed from the valve assembly following its connection to the valve assembly. By creating a positive displacement of fluid from the catheter and preventing reflux into the catheter, the risk of blocking the flow path by clotting is substantially reduced without the use of additional drugs.

The valve assembly includes an housing having a first portion proximal to a needle protector, and a second portion distal to the needle protector; the first portion including one or more flow channels in flow communication with the second portion to direct fluid around a resilient septum; the second portion including a seat for retaining an internal part of the resilient septum, and one or more air vents; and the resilient septum defining a hollow therein, the resilient septum located within the housing and having a first end positioned against an actuator, the actuator having a plurality of slots in flow communication with one or more flow channels, a stepped second end for seating the resilient septum on the seat in the second portion.

The valve assembly includes an housing having a connection end proximal to a needle protector, a catheter end distal to the needle protector, and a middle portion between the connection end and the catheter end; the connection end having a female luer for receiving the needleless device; the middle portion including an actuator, a resilient septum, a flow channel in flow communication with the connection end to direct fluid around the resilient septum, and an expansion chamber; the catheter end including one or more flow channels in flow communication with an intravenous catheter, and an air vent in communication with the expansion chamber; and the resilient septum defining a hollow therein, the resilient septum located within the housing and having a first end positioned against the actuator, the actuator having a plurality of slots in flow communication with the flow channel, a stepped second end for seating the resilient septum on the seat in the middle portion.

### DESCRIPTION OF THE DRAWINGS

FIG. 1 is a perspective view of the catheter apparatus of an embodiment of the present invention.

FIG. 2 is a cross-sectional view of the catheter apparatus of an embodiment of the present invention shown with the actuator in a first position.

FIG. 3 is a cross-sectional view of the catheter apparatus of FIG. 2 shown with the actuator in a second position.

FIG. 3A is an expanded view of the actuator and flow path through the actuator of the catheter apparatus of FIG. 3.

FIG. 4 is a perspective view of a catheter hub according to an embodiment of the present invention.

FIG. 5 is a cross-sectional view of the catheter hub of FIG. 4 taken along line 2-2 of FIG. 4 showing the actuator in a first position.

FIG. 6 is a cross-sectional view of the catheter hub of FIG. 4 taken along line 2-2 of FIG. 4 showing a single flow channel.

FIG. 7 is a cross-sectional view of the catheter hub of FIG. 4 showing the actuator in a second position.

FIG. 8 is a further cross-sectional view of the catheter hub of FIG. 4 showing the actuator in a second position and the air vents.

FIG. 9 is a cross-sectional view of the catheter hub of FIG. 4 showing a single air vent.

FIG. 10 is an exploded view of FIG. 4 showing the components of the catheter hub.

FIG. 11 is a sectional view of the housing of FIG. 4 showing the flow channels that allow fluid to flow around the resilient septum.

FIG. 12 is an isometric view of the housing of FIG. 4 showing the flow channels and air vents.

FIG. 13 is a section of FIG. 12 showing the face where the resilient septum is seated, the internal compartment of the air vents, and the flow channels.

FIG. 14 is an isometric view of another embodiment of the present invention.

FIG. 15 is a cross-sectional view of FIG. 14 showing the actuator in a first position.

FIG. 16 is a cross-sectional view of FIG. 14 showing more than one flow channel in the catheter end.

FIG. 17 is a cross-sectional view of FIG. 14 showing more than one flow channel in the middle section.

FIG. 18 is a cross-sectional view of FIG. 14 showing the actuator in a second position.

FIG. 19 is an exploded view of FIG. 14 showing the components of the housing.

### DETAILED DESCRIPTION OF THE INVENTION

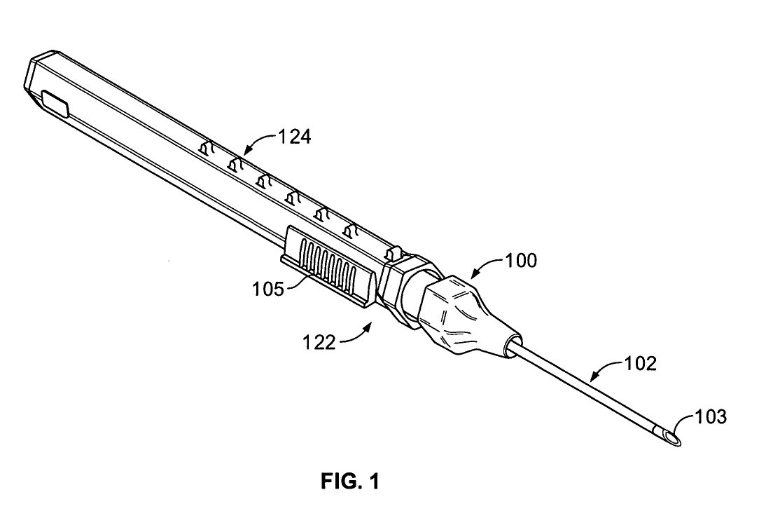

As shown in FIG. 1, the intravenous catheter assembly 122 of the present invention has a needle protector 124, a catheter hub 100, an over-the-needle plastic catheter tubing 102, and a hollow bore needle 103. The needle protector 124 connects to the catheter hub 100 using a mating luer system of threaded interlocking pieces. These threads are typically constructed to conform to American National Standard Institute No. ANSI/HIMA MD70.1-1983 or ISO 594/2-1998 relating to luer lock fittings. Other connection systems, however, may be used to connect the needle protector 124 and the catheter hub 100 without departing from the spirit and scope of the present invention.

One using the intravenous catheter assembly 122 locates a blood vessel on the patient's body. The needle 103 and catheter tubing 102 are inserted through the skin and blood vessel of the patient. Once the needle is in the blood vessel, blood “flashes” through the needle fluid passageway or catheter tubing 102. The needle 103 is removed from the intravenous catheter assembly 122 by sliding the ridge portion of the sliding needle hub 105 along the sides of the needle protector 124 away from the catheter hub 100. This causes the needle 103 to be removed from the catheter hub 100 into the needle protector 124, where it is locked into place to prevent accidental needle sticks. Once the needle 103 is secured within the needle protector 124, the needle protector 124 can be removed from the intravenous catheter assembly 122 and discarded. After removal of the needle 103 from the blood vessel, the catheter tubing 102 remains positioned in the blood vessel. With the needle protector 124 removed, the catheter hub 100 of the intravenous catheter assembly 122 can receive a needleless device using the connection system already in place. This could be for example, a needleless device having a mating luer that locks with the luer lock fitting on the catheter hub 100.

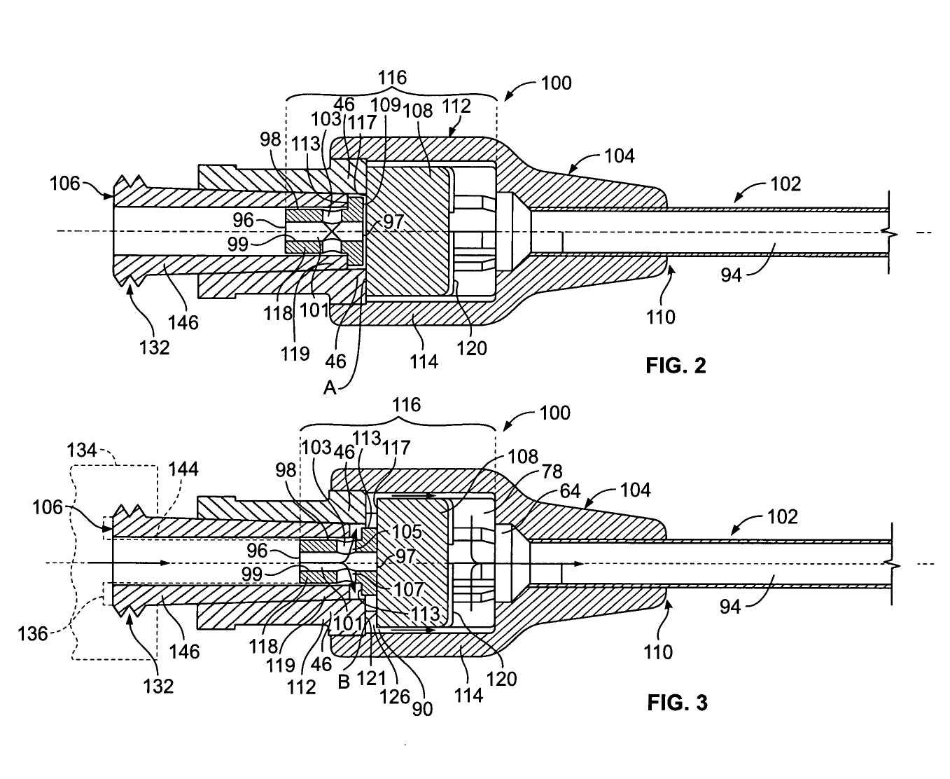

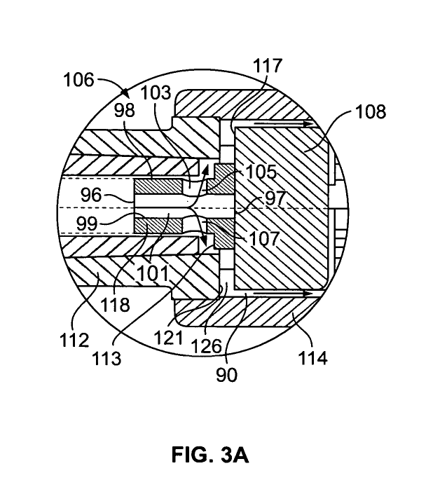

As shown in greater detail in FIGS. 2 and 3, the catheter hub 100 includes a housing 104 having a connection end 106 and a catheter end 110 together defining a flow path 126. The housing 104 includes a plurality of walls 114 arranged in a geometric configuration or alternatively may include a hub wall in a circular configuration. A valve assembly 116 is positioned in the housing 104 for regulating fluid flow through the flow path 126 between a luer of a needleless device 134 and the catheter tubing 102. The valve assembly includes a body 112, a septum 108 and an actuator 118. The septum 108 is made of a resilient, compressible elastomeric material. The resilient, compressible elastomeric material includes, but is not limited to, natural and/or synthetic elastomers such as silicones, polyisoprenes, thermoplastic vulcanates, or a combination thereof.

As shown in FIG. 3, the connection end 106 of the housing 104 contains a luer receiving portion 146 into which the luer of the needleless device 134 is received. The luer receiving portion 146 contains luer lock projections 132 that are complementary to luer lock recesses or threads 136 of the luer of the needleless device 134. The needleless device (not shown) also contains a male member 144 that, when inserted into the connection end 106 of the housing 104, engages the actuator 118 of the valve assembly 116.

FIGS. 2, 3, and 3A show an actuator 118 including a first actuator end 96, a second actuator end 97, an exterior actuator surface 98, and an interior actuator surface 99. The interior actuator surface 99 defines an actuator fluid passage way 101 extending between the first actuator end 96 and the second actuator end 97. The actuator 118 further includes second fluid passageways 103 and 107 that extend perpendicular to the first actuator fluid passageway 101. The second fluid passageways 103 and 107 consist of openings 105 and 107 that each extends from the interior actuator surface 99 to the exterior actuator surface 98. The actuator exterior surface 98 defines an annular septum contact surface 109 and an opposed actuator shoulder contact surface 113. The septum contact surface 109 engages the shoulder surface 117 of the septum 108. The actuator shoulder contact surface 113 engages the actuator shoulder 119 of the body 112.

FIG. 2 shows the actuator 118 in a first position “A” where a seal is formed between the shoulder surface 117 (as shown in FIG. 3) of the septum 108 and septum shoulder 121 (as shown in FIG. 3) of the body 112. The shoulder surface 117 engages the septum shoulder 46 defined by the body 112 to form a seal when the valve assembly 116 is in a sealed position. The seal is tight because the shoulder surface 117 of the septum 108 is forced against the septum shoulder 46 due to the resilient nature of the septum. Therefore, blood or other fluids will be prevented by the seal from escaping from the device.

FIG. 3 shows the actuator 118 in a second position “B” when the male member 144 of the needleless device is inserted into the connection end 106 of the housing 104, causing the male member 144 to engage the actuator end 96 of the actuator 118. FIG. 3A shows an expanded view of the actuator 118 and flow path through the actuator when the actuator 118 is in the second position “B.” The engagement of the actuator end 96 by the male member 144 causes the septum contact surface 109 of the actuator 118 to engage and press against the shoulder surface 117 of the septum 108. Due to the resilient nature of the septum 108, the shoulder surface 117 becomes disengaged from the septum shoulder 46 of the body 112. This breaks the seal between the shoulder surface 117 and the septum shoulder 46. As shown by the arrows in FIG. 3, fluid is free to flow from the luer of the needleless device 134 through the first actuator fluid passageway 101 to the second actuator fluid passageways 103 and 107 through the chamber fluid passageways 126 and 90 through the support structure 120 to the channels 78 through the eyelit fluid passageway 64 and to the catheter fluid passageway 94 into the blood vessel. Fluid can also flow in the opposite direction.

As shown in FIGS. 2 & 3, the resilient septum 108 is supported toward the catheter end 110 end by a support structure 120. The support structure 120 is comprised of a substantially inflexible material and has a plurality of openings to allow the passage of fluid through the support structure 120. The support structure 120 may be manufactured from any suitable material, for example, a plastic or metal in the form of a grate or other structure that includes openings. This positioning prevents the resilient septum 108 from deforming into the flow path 126 when the actuator 118 is actuated. When the actuator 118 is moved from the first position “A” to the second position “B”, the resilient septum 108 is compressed and the support structure 120 prevents the resilient septum 108 from moving toward the catheter end 110. Controlling the deformation of the resilient septum 108 when actuated is important so that the deformation of the resilient material does not impede the flow paths 126 and 90.

The valve assembly 116 can be resealed by removing the luer of the needleless device 134 from the connection end 106 of the body 112 thereby causing the septum 108 to regain its original, as-assembled shape to form a seal between the shoulder surface 117 and the septum shoulder 46. The luer of the needleless device 134 is removed from the catheter hub 100 by rotating the luer lock fitting 136 in the opposite direction to that used to engage the fitting 136 to the luer attachment fitting 132. This action causes the resilient septum 108 to regain its original, as-assembled shape and return the actuator 118 to the first position A to form a seal between the housing 104 and the resilient septum 108. When the luer of the needleless device 134 is removed from the connection end 106, the flow path 126 volume decreases, resulting in an ejection of fluid into the catheter tubing 102. The flow path decreases because the septum 108 expands when the luer of the needleless device 134 is removed.

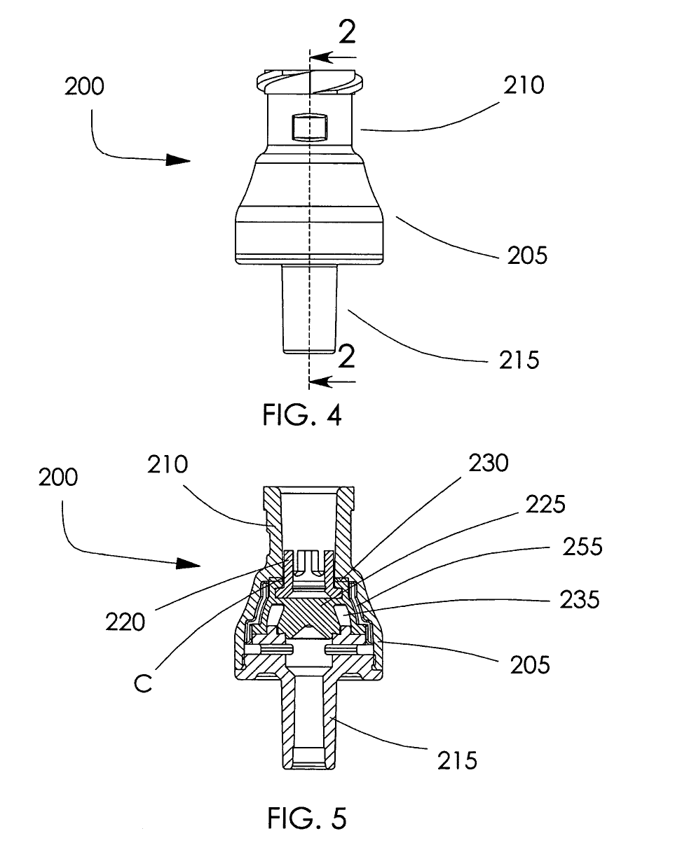

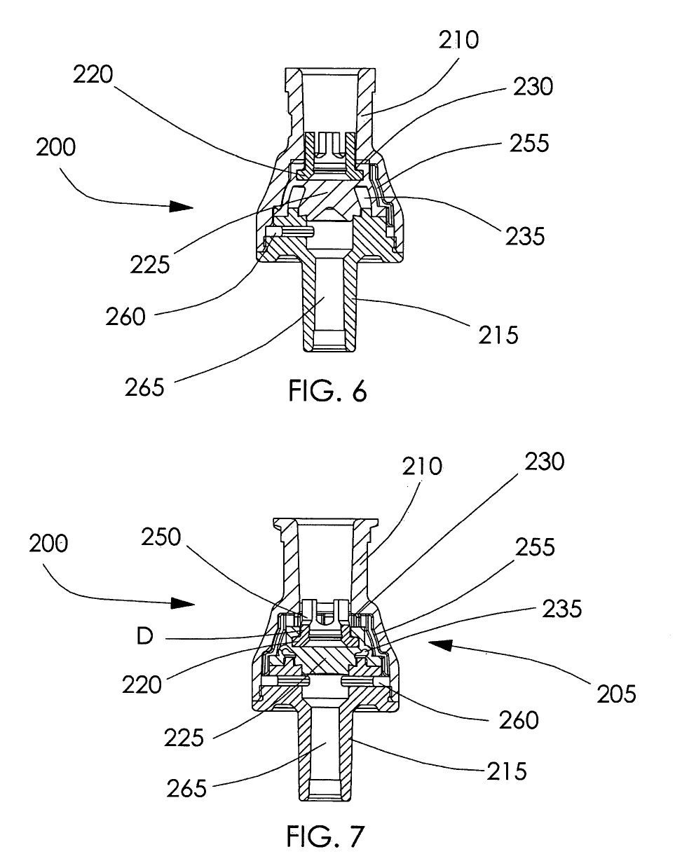

FIGS. 4-13 depict another embodiment of the present invention. FIG. 4 illustrates a catheter hub 200. The catheter hub 200 includes a housing 205 having a first portion 210 proximal to a needle protector (not shown) and a second portion 215 distal to the needle protector. As depicted in FIG. 5, the housing 205 contains an actuator 220 located proximal to the first portion 210 and a resilient septum 225 located adjacent to the actuator 220. The resilient septum 225 is sealed against the first portion 210 at face seal 230. The resilient septum 225 is made of a resilient, elastomeric, generally incompressible material, including, but not limited to, polyisoprene or silicone. The resilient septum 225 defines at least one hollow 235 such as an air pocket. The actuator 220 is shown in a first position “C” in FIG. 5. FIG. 5 also depicts various flow channels 255 through which fluid flows when a needleless device (not shown) containing fluid is attached to the first portion 210 and depresses the actuator 220 against the resilient septum 225. As shown in FIG. 6, instead of various flow channels, the embodiment may also contain a single flow channel 255 in the first portion 210 and a single flow channel 260 in the second portion 215 of the catheter hub 200.

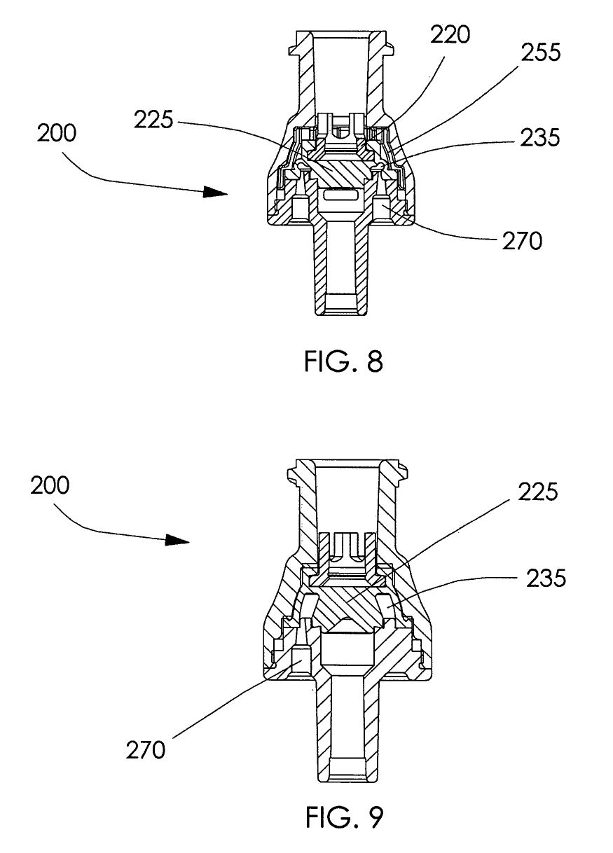

FIG. 7 illustrates the actuator 220 in a second position “D” when a needleless device, such as a male luer (not shown), is attached to the first portion 210 of the catheter hub 200. The actuator 220 is pushed down by the male luer so that cutouts 250 are below the face seal 230. Fluid then flows through the cutouts 250, down various flow channels 255 in the first portion 210, through flow channels 260 in the second portion 215, and then down through the tubular portion 265 of the second portion 215. As the resilient septum 225 is compressed by the actuator 220, the hollow 235 is compressed, decreasing the air volume in the resilient septum 225. As shown in FIG. 8, air is vented to the exterior of the catheter hub 200 by air vents 270 when the resilient septum 225 is compressed by the actuator 220. When the resilient septum 225 is activated, the volume increases in the various flow channels 255. Upon removal of a needleless device, this volume decreases, creating a positive displacement of fluid out the second portion 215. FIG. 9 shows an embodiment with only one air vent 270.

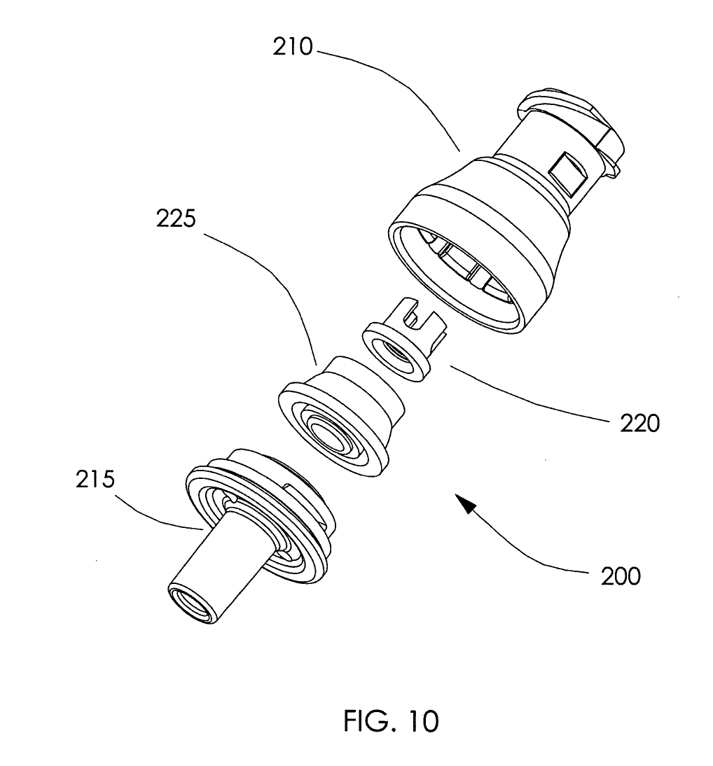

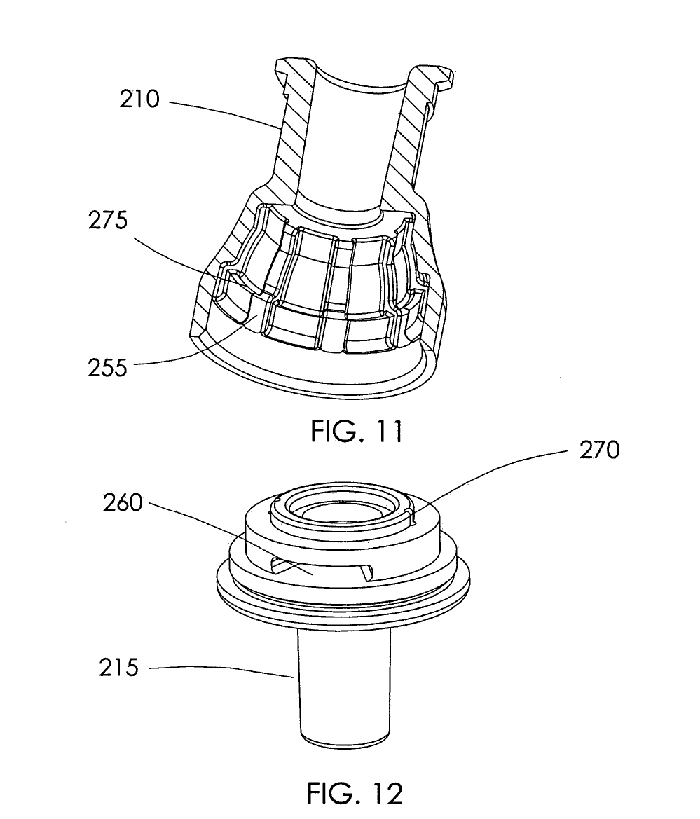

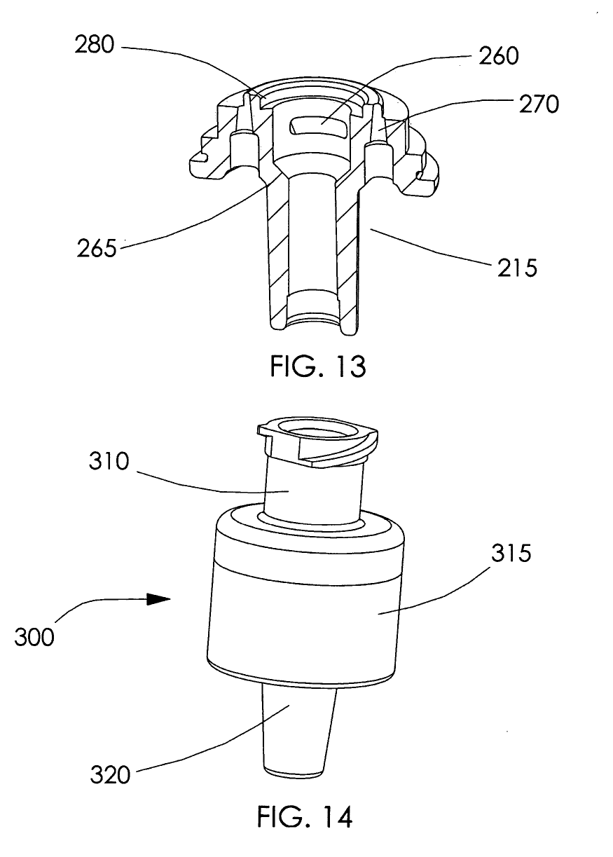

FIG. 10 shows an exploded perspective view of the first portion 210, the actuator 220, the resilient septum 225, and the second portion 215 of the catheter hub 200. FIG. 11 is a cut-away view showing the flow channels 255 in the first portion 210 of the catheter hub 200 through which fluid flows around the resilient septum 225 when the actuator 220 is in the second position D as shown in FIG. 7. As shown in FIG. 1, the housing contains an energy director 275 that facilitates a seal between the resilient septum 225 and the housing of the second portion 215. FIG. 12 depicts the flow channel 260 and the air vent 270 in the second portion 215 of the catheter hub 200. FIG. 13 is a cross sectional view of FIG. 12 showing the internal area of the air vent 270 and flow channel 260 in the second portion 215 of the catheter hub 200. FIG. 13 also illustrates the face 280 where the resilient septum 225 is seated against the second portion 215.

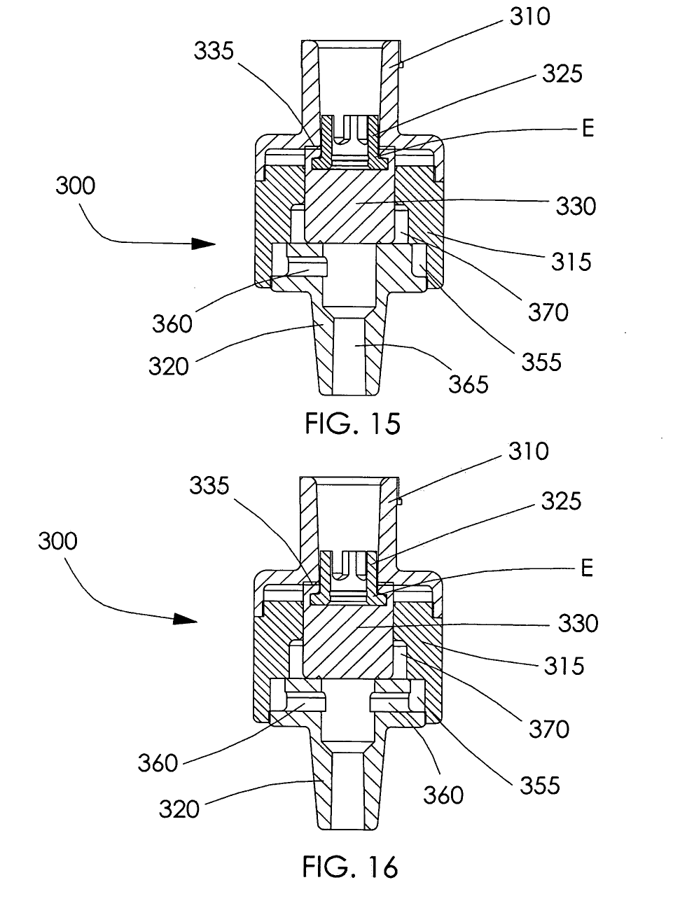

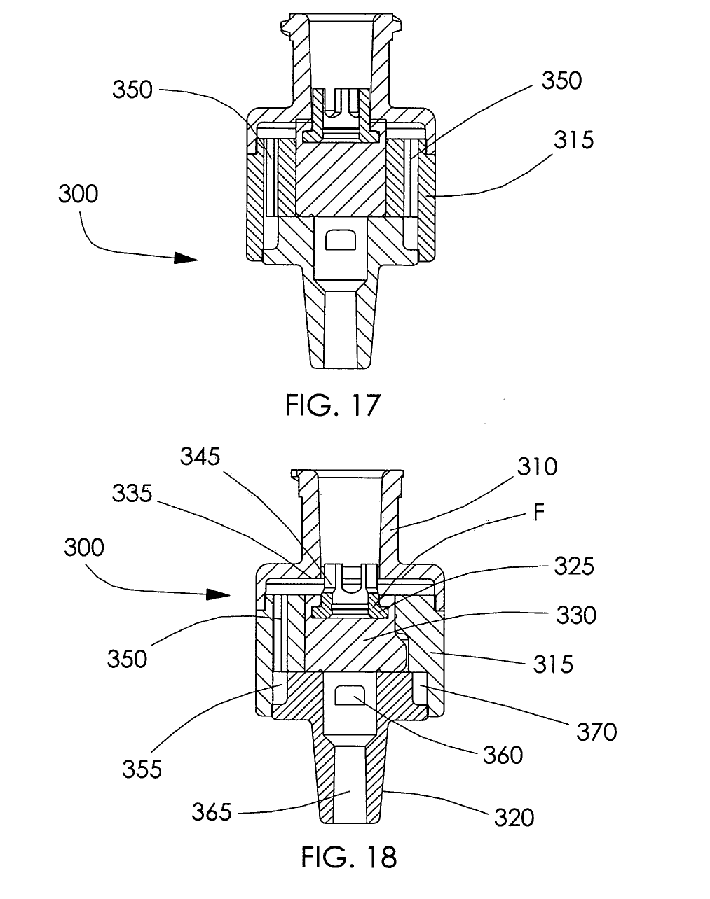

FIGS. 14-19 illustrate another embodiment of the present invention. FIG. 14 shows an isometric view of a catheter hub 300 having a connection end 310, a middle section 315, and a catheter end 320. As shown in FIG. 15, the catheter hub 300 contains an actuator 325 located proximal to the connection end 310 and a resilient septum 330 located adjacent to the actuator 325. The actuator 325 is shown at a first position E in FIG. 15. The resilient septum 330 is sealed against the connection end 310 at face seal 335. The resilient septum 330 is made of an elastomeric, generally incompressible material, preferably, but not limited to, polyisoprene or silicone. Adjacent to the resilient septum 330 is at least one rigid expansion chamber 370. FIG. 15 also depicts a flow channel 360 in the catheter end 320 which facilitates the flow of fluid from the flow channel 350 in the middle section 315 (shown in FIG. 18) into the tubular portion 365 of the catheter end 320. As shown in FIG. 16, the catheter end 320 may also contain more than one flow channel 360. FIG. 17 shows the embodiment having more than one flow channel 350 through the middle section 315.

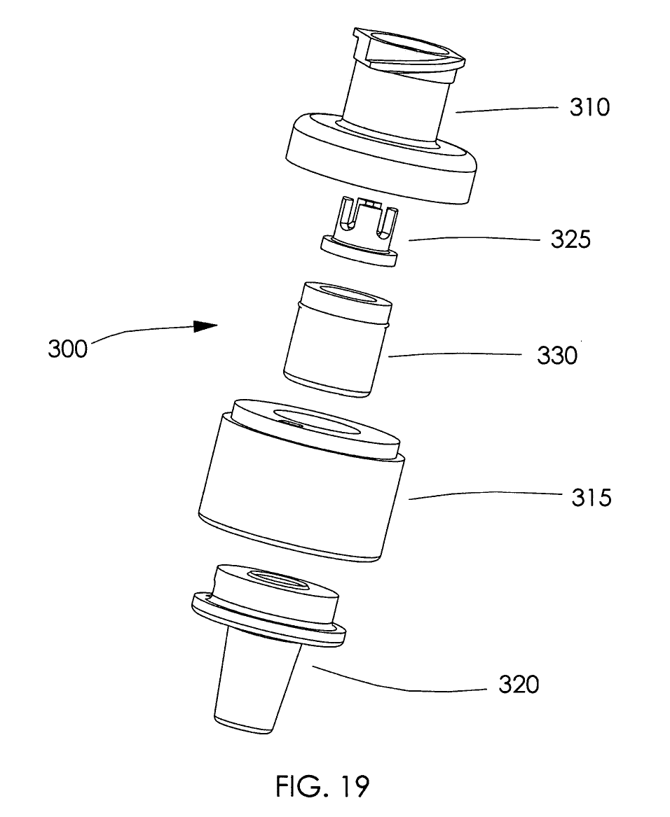

FIG. 18 shows the actuator 325 in a second position F when a needleless device, such as a male luer (not shown), is attached to the connection end 310 of the catheter hub 300. The actuator 325 is pushed down by the male luer so that cutouts 345 are below the face seal 335. Fluid then flows through the cutouts 345, at least one flow channel 350 in the middle section 315, around the annular volume 355, into at least one flow channel 360 in the catheter end 320, and then down through a tubular portion 365 of the catheter end 320. As the resilient septum 330 is compressed by the actuator 325, the resilient septum 330 bulges into the expansion chamber 370. The air displaced by the resilient septum in the expansion chamber 370 may be vented to the exterior of the catheter hub 300 by one or more air vents (not shown). When the resilient septum 330 is activated, the volume increases above it, in communication with the flow channels 350 and 360. Upon removal of a needleless device, this volume decreases, creating a positive displacement of fluid out the tubular portion 365 of the catheter end 320. FIG. 19 shows a further view of the connection end 310, the actuator 325, the resilient septum 330, the middle section 315, and the catheter end 320 of the catheter hub 300.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed:

1. A valve assembly for an intravenous catheter comprising:
a housing;
an actuator located within the housing;
a resilient septum adjacent to the actuator; and
means for creating a positive displacement of fluid from the intravenous catheter when a needleless device is removed from the valve assembly following its connection to the valve assembly.

2. The valve assembly of claim 1, wherein the means is comprised of a support structure located proximal to the resilient septum.

3. The valve assembly of claim 2, wherein the resilient septum comprises a compressible material.

4. The valve assembly of claim 3, wherein the compressible material is silicone.

5. The valve assembly of claim 2, wherein the support structure is a plastic grate.

6. The valve assembly of claim 2, wherein the support structure is a metal grate.

7. The valve assembly of claim 1, wherein:
the housing has a first portion proximal to a needle protector, and a second portion distal to the needle protector;
the first portion including one or more flow channels in flow communication with the second portion to direct fluid around the resilient septum;
the second portion including a seat for retaining an internal part of the resilient septum, and one or more air vents; and
the resilient septum defines a hollow therein, the resilient septum located within the housing and having a first end positioned against the actuator, the actuator having a plurality of slots in flow communication with one or more flow channels, a stepped second end for seating the resilient septum on the seat in the second portion.

8. The valve assembly of claim 7, wherein the hollow comprises an air pocket encompassed by the resilient septum.

9. The valve assembly of claim 8, wherein the air pocket is vented to an area exterior to the valve assembly.

10. The valve assembly of claim 7, wherein the resilient septum comprises a generally incompressible material.

11. The valve assembly of claim 10, wherein the generally incompressible material is silicone.

12. The valve assembly of claim 10, wherein the generally incompressible material is synthetic polyisoprene.

13. The valve assembly of claim 1, wherein:
the housing has a connection end proximal to a needle protector, a catheter end distal to the needle protector, and a middle portion between the connection end and the catheter end;
the connection end having a female luer for receiving the needleless device;
the middle portion including the actuator, the resilient septum, a flow channel in flow communication with the connection end to direct fluid around the resilient septum, and an expansion chamber;
the catheter end including one or more flow channels in flow communication with an intravenous catheter, and an air vent in communication with the expansion chamber;
the resilient septum defines a hollow therein, the resilient septum located within the housing and having a first end positioned against the actuator, the actuator having a plurality of slots in flow communication with a flow channel, a stepped second end for seating the resilient septum on the seat in the middle portion.

14. The valve assembly of claim 13, wherein a portion of the resilient septum bulges into the expansion chamber when the actuator is compressed by the needleless device.

15. The valve assembly of claim 13, wherein the resilient septum comprises a generally incompressible material.

16. The valve assembly of claim 15, wherein the resilient septum is silicone.

17. The valve assembly of claim 15, wherein the resilient septum is synthetic polyisoprene.

18. A housing assembly for an insertion device catheter, the housing assembly comprising:
a housing having a catheter end and a connection end, said housing defining a flow path extending between said catheter and connection ends;
a valve assembly positioned in said flow path in sealing engagement with said housing, said valve having a substantially solid, resilient component; and
a support structure positioned proximal to the catheter end within the housing.

19. The housing assembly of claim 18, wherein the valve assembly comprises an actuator and a resilient septum.

20. The housing assembly of claim 18, wherein the support structure includes a plurality of openings through which fluid may pass.

21. The housing assembly of claim 18, wherein the support structure is comprised of a substantially solid material.

22. The housing assembly of claim 21, wherein the support structure is comprised of plastic.

23. The housing assembly of claim 18 wherein the housing assembly is used in connection with a needleless device.

24. A catheter assembly comprising:
a needle protector;
a catheter apparatus; and
a catheter hub comprising:
a housing having a catheter end and a connection end, said housing defining a flow path extending between said catheter and connection ends;
a valve assembly positioned in said flow path in sealing engagement with said housing; and
a support structure positioned proximal to the catheter end within the housing.

25. The catheter assembly of claim 24, wherein the support structure further includes a plurality of openings through which fluid may pass.

26. The catheter assembly of claim 24, wherein the support structure is comprised of a substantially solid material.

27. The catheter assembly of claim 26, wherein the support structure is comprised of plastic.

28. A catheter assembly comprising:
a needle protector;
a catheter apparatus; and
a valve assembly comprising a housing, an actuator, a resilient septum, and a flow path, wherein the resilient septum contains at least one compressible air pocket.

29. The catheter assembly of claim 28, wherein the air pocket is vented to the exterior of the housing.

30. A catheter apparatus comprising:
a needle protector;
a catheter assembly; and
a valve assembly comprising a housing, an actuator, a resilient septum, and a rigid expansion chamber.

31. The catheter apparatus of claim 30, wherein the rigid expansion chamber is vented to the exterior of the housing.

32. The catheter apparatus of claim 31, wherein a portion of the resilient septum expands into the rigid expansion chamber when the actuator is depressed by a needleless device.
